(19) United States
(12) Patent Application Publication
(10) Pub. No.: US 2022/0146523 A1
(43) Pub. Date: May 12, 2022

(54) RENAL CLEARABLE NANOPARTICLES AS EXOGENOUS MARKERS FOR EVALUATING KIDNEY FUNCTION

(71) Applicant: Board of Regents, The University of Texas System, Austin, TX (US)

(72) Inventors: Jie ZHENG, Plano, TX (US); Mengxiao YU, Frisco, TX (US); Xuhui NING, Richardson, TX (US)

(21) Appl. No.: 17/434,132

(22) PCT Filed: Feb. 26, 2020
(86) PCT No.: PCT/US2020/019782

§ 371 Date: Aug. 26, 2021

Related U.S. Application Data
(60) Provisional application No. 62/810,626, filed on Feb. 26, 2019.

(51) Int. Cl. G01N 33/58; A61K 49/00

(52) U.S. Cl. CPC G01N 33/587; G01N 2800/347; A61K 49/0004; G01N 33/582

(57) ABSTRACT

A method for evaluating kidney function utilizing a nanoparticle that can be eliminated from the body by the kidneys as an exogenous marker. The method includes administering the nanoparticles to a subject, followed by collecting a blood or urine sample after a period of time, characterizing the nanoparticles in the blood or urine sample, and finally comparing a characteristic parameter of the nanoparticles in the blood or urine sample between the tested subject and a control group having normal kidney function.

### RELATED APPLICATIONS

This application claims priority to, and the benefit of, U.S. Provisional Application No. 62/810,626, filed Feb. 26, 2019, the contents of which are incorporated herein by reference in their entireties.

### GOVERNMENT SUPPORT

This invention was made with government support under Grant No. R43 DK116368 awarded by the National Institutes of Health. The government has certain rights in the invention.

### FIELD OF THE DISCLOSURE

This disclosure relates to the evaluation and diagnosis of kidney disease, such as early detection of kidney dysfunction in living subjects utilizing nanoparticles that can be eliminated from the body by the kidneys as an exogenous marker.

### BACKGROUND

Kidney disease affects more than 10% of the population worldwide (850 million), which is twice the number of people living with diabetes (422 million) and 20 times the number of cancer patients (42 million). Chronic kidney disease (CKD) causes 1.2 million deaths in the world each year, more than breast and prostate cancers combined. In the United States, about 30 million people (15% of adults) are living with CKD that can be caused by diabetes, high blood pressure, glomerulonephritis, cystic kidney disease and other disorders. Different from CKD wherein kidney function is gradually lost over several months or years, acute kidney injury (AKI) is characterized by an abrupt reduction in kidney function within a few hours or days. AKI is a deadly disease and a common clinical complication affecting 13.3 million patients worldwide annually, including 3.2-20% of all hospitalized patients and 22-67% intensive care unit (ICU) patients. Despite supportive care including renal replacement therapy, the 5-year mortality after AKI remains over 50%. In addition, many medicines can induce AKI due to drug-induced nephrotoxicity, such as cisplatin (a chemotherapeutic agent for cancer treatment). Moreover, patients with systemic lupus erythematosus (SLE, lupus, a chronic autoimmune disease) also have a high risk in developing a type of kidney disease—lupus nephritis, a life-threatening complication due to renal deposition of immune complexes. At least 5 million people worldwide and 1.5 million Americans suffer from lupus and over 90% of them are women between the ages of 15-44. While SLE is a systemic disease that affects multiple organs, kidney involvement is a leading cause of morbidity and mortality in SLE: generally, about 60% of lupus patients will develop lupus nephritis during their lives and have a higher mortality than those without kidney involvement.

As a “silent killer,” kidney disease often has no signs or symptoms in the early stages and remains undetected until it is very advanced. For instance, approximately 60% of CKD cases get diagnosed only at the end-stage renal disease (ESRD, more than 85% of normal kidney function is lost), which is fatal without dialysis or a kidney transplant. Within 15 years of diagnosis, 10%-30% of lupus nephritis patients progress to ESRD even with treatment. The treatment for ESRD consumes $30.9 billion to care in the United States in 2013, about 7.1 percent of the overall Medicare paid claims costs.

While kidney disease is usually a progressive disease and the decline of kidney function is unavoidable, kidney disease can be managed effectively if it is diagnosed in the early stages before irreversible damages have occurred. For example, drug-induced AKI can often be cured if diagnosed and treated early. For CKD that cannot be cured, early diagnosis and management is crucial not only to delay or prevent progression to ESRD, but also to lower the risk for heart disease and stroke. Therefore, for people who have risk factors for CKD, including diabetes, high blood pressure, heart disease, obesity, and a family history of CKD, it is recommended that kidney function be checked regularly (e.g., every 3 or 6 months) with blood and urine tests. These tests measure endogenous biomarkers such as blood urea nitrogen (BUN), serum creatinine, and urine protein. Lupus nephritis also has no cure. Early diagnosis of kidney involvement and treatment of lupus nephritis is critical to stop kidney damage early so that kidney failure can be postponed or even prevented. Therefore, the kidney function of lupus patients needs to be monitored every 3 months (in average) with blood and urine tests in order to catch lupus nephritis at an early stage. Since proteinuria is more sensitive than serum creatinine in identifying lupus nephritis, once proteinuria is detected, the patients will be subjected to kidney biopsy that is the gold standard for classifying the lupus nephritis and guiding the treatment options.

In addition to early diagnosis of kidney disease, accurate monitoring of kidney function is also necessary during the treatment of kidney disease. For example, lupus nephritis is usually treated with immunosuppressive agents: on one hand, the response to immunosuppressive drugs is quite variable among patients and only ˜50% of patients responded to treatment in some large trial; on the other hand, immunosuppressive drugs have many potential side effects, such as lowering the blood counts and increasing risks for infection and cancer. Thus, during the treatment of lupus nephritis, doctors need to closely monitor the kidney function of patients (every 1 month in average) so that they can optimize the use of medications to achieve remission of the disease while reducing the side effects. Moreover, monitoring of kidney function can bring awareness to CKD patients on their diets to prevent further damage to the kidneys.

Despite the clinical need, early diagnosis of kidney dysfunction and accurate monitoring of kidney function remain challenging for current methods. Thus, there remains a need in the art for a method for diagnosing the dysfunction of a live kidney in the early stages (or evaluating the function of a live kidney) that is not only simple, affordable, and widely accessible, but also is highly sensitive and accurate. The present disclosure is provided to address this need and offer advantages not provided by prior diagnostic techniques.

### SUMMARY

The present disclosure provides, inter alia, methods of evaluating or monitoring kidney function of a subject using renal clearable nanoparticles.

In one aspect, the present disclosure provides a method of evaluating kidney function of a subject, the method comprising: (a) administering to the subject a first plurality of nanoparticles having a first dose; (b) collecting a urine sample and/or a blood sample from the subject after a first period of time after the administration; (c) characterizing the nanoparticles in the urine sample and/or the blood sample with a measurement process to obtain a characteristic parameter; and (d) comparing the characteristic parameter with a control characteristic parameter measured for a control group having normal kidney function, thereby evaluating the kidney function.

In another aspect, the present disclosure provides a method of evaluating kidney function of a subject, the method comprising: (c) characterizing a first plurality of nanoparticles in a urine sample and/or a blood sample collected after a first period of time from the subject administered with the first plurality of nanoparticles, with a measurement process to obtain a characteristic parameter; and (d) comparing the characteristic parameter with a control characteristic parameter measured for a control group having normal kidney function, thereby evaluating the kidney function.

In some embodiments of any one of the above aspects, the control characteristic parameter is measured by: (e) administering to the control group a second plurality of the nanoparticles having a second dose; (f) collecting a urine sample and/or a blood sample from the control group after the first period of time after the administration; and (g) characterizing the nanoparticles in the urine sample and/or the blood sample with the measurement process to obtain the control characteristic parameter.

In another aspect, the present disclosure provides a method of evaluating kidney function of a subject, the method comprising: (a) administering to the subject a first plurality of nanoparticles having a first dose; (b) collecting a urine sample and/or a blood sample from the subject after a first period of time after the administration; (c) characterizing the nanoparticles in the urine sample and/or the blood sample with a measurement process to obtain a characteristic parameter; and (d) comparing the characteristic parameter with a reference value, thereby evaluating the kidney function.

In another aspect, the present disclosure provides a method of evaluating kidney function of a subject, the method comprising: (c) characterizing a first plurality of nanoparticles in a urine sample and/or a blood sample collected after a first period of time from the subject administered with the first plurality of nanoparticles, with a measurement process to obtain a characteristic parameter; and (d) comparing the characteristic parameter with a reference value, thereby evaluating the kidney function.

In yet another aspect, the present disclosure provides a method of monitoring kidney function of a subject, the method comprising: (a) administering to the subject a first plurality of nanoparticles having a first dose; (b) collecting a first urine sample and/or a first blood sample from the subject after a first period of time after the administration of step (a); (c) characterizing the nanoparticles in the first urine sample and/or the first blood sample with a measurement process to obtain a first characteristic parameter; (d) after a second period of time after step (b), administering to the subject a second plurality of the nanoparticles having a second dose; (e) collecting a second urine sample and/or a second blood sample from the subject after a third period of time after the administration of step (d); (f) characterizing the nanoparticles in the second urine sample and/or the second blood sample with the measurement process to obtain a second characteristic parameter; and (g) comparing the first characteristic parameter with the second characteristic parameter, thereby monitoring the kidney function over time.

In yet another aspect, the present disclosure provides a method of monitoring kidney function of a subject, the method comprising: (c) characterizing a first plurality of nanoparticles in a first urine sample and/or a first blood sample collected after a first period of time from the subject administered with the first plurality of nanoparticles, with a measurement process to obtain a first characteristic parameter; (d) after a second period of time after step (c), characterizing a second plurality of nanoparticles in a second urine sample and/or a second blood sample collected after a third period of time from the subject administered with the second plurality of nanoparticles, with the measurement process to obtain a second characteristic parameter; and (e) comparing the first characteristic parameter with the second characteristic parameter, thereby monitoring the kidney function over time.

In some embodiments of any one of the above aspects, the method further comprises indicating kidney dysfunction or injury when the characteristic parameter is significantly different from the control characteristic parameter or reference value.

In some embodiments of any one of the above aspects, the nanoparticles are renal clearable.

In some embodiments of any one of the above aspects, the nanoparticles have a 1-hour or 2-hour renal clearance efficiency in the range of 5 to 100 percent of injected dose (% ID). In some embodiments of any one of the above aspects, the nanoparticles have a 1-hour renal clearance efficiency in the range of 5 to 100 percent of injected dose (% ID).

In some embodiments of any one of the above aspects, the nanoparticles comprise gold, silver, copper, platinum, palladium, silica, carbon, silicon, iron oxide, FeS, CdSe, CdS, CuS, an organic material, or a combination thereof.

In some embodiments of any one of the above aspects, the nanoparticles are coated with a ligand selected from the group consisting of glutathione, thiol-functionalized polyethylene glycol, cysteamine, cysteine, homocysteine, a dipeptide containing cysteine, a dipeptide containing homocysteine, a peptide having more than three amino acids, and a combination thereof.

In some embodiments of any one of the above aspects, the dipeptide containing cysteine includes cysteine-glycine or cysteine-glutamic acid.

In some embodiments of any one of the above aspects, the dipeptide containing homocysteine includes homocysteine-glycine or homocysteine-glutamic acid.

In some embodiments of any one of the above aspects, the ligand is conjugated with a fluorescent dye.

In some embodiments of any one of the above aspects, the ligand is glutathione.

In some embodiments of any one of the above aspects, the nanoparticles fluoresce in a range of 500 to 850 nm.

In some embodiments of any one of the above aspects, the nanoparticles fluoresce in a range of 1000 to 1700 nm.

In some embodiments of any one of the above aspects, the first period of time is about 30 minutes to 24 hours for the urine sample, or about 5 mins to 1 hour for the blood sample.

In some embodiments of any one of the above aspects, the third period of time is about 30 minutes to 24 hours for the urine sample, or about 5 mins to 24 hours for the blood sample.

In some embodiments of any one of the above aspects, the first period of time and the third period of time are the same.

In some embodiments of any one of the above aspects, the first period of time and the third period of time are different.

In some embodiments of any one of the above aspects, the measurement process includes one or more processes selected from the group consisting of: measuring a concentration of the nanoparticles, measuring an amount of the nanoparticles, measuring clearance efficiency, analyzing a composition of ligands on the nanoparticle surface, measuring size distribution of the nanoparticles, measuring an absorption spectrum of the nanoparticles, measuring an emission spectrum of the nanoparticles, measuring an excitation spectrum of the nanoparticles, measuring a photoacoustic signal of the nanoparticles, measuring radioactivity of the nanoparticles, or a combination thereof.

In some embodiments of any one of the above aspects, the measurement process includes inductively coupled plasma mass spectrometry (ICP-MS) or inductively coupled plasma optical emission spectroscopy (ICP-OES).

In some embodiments of any one of the above aspects, the characteristic parameter is selected from the group consisting of: a concentration of the nanoparticles, an amount of the nanoparticles, clearance efficiency, a composition of ligands on the nanoparticle surface, one type of surface ligand, size distribution of the nanoparticles, surface charge of the nanoparticles, an absorption spectrum of the nanoparticles, an emission spectrum of the nanoparticles, an excitation spectrum of the nanoparticles, a photoacoustic signal of the nanoparticles, radioactivity of the nanoparticles, and a combination thereof.

In some embodiments of any one of the above aspects, the characteristic parameter is the concentration of the nanoparticles in the blood sample of the subject; the control characteristic parameter is the control concentration of the nanoparticles in the blood sample of the control group; and if the concentration is significantly different from the control concentration, then it indicates kidney dysfunction or injury.

In some embodiments of any one of the above aspects, the characteristic parameter is the clearance efficiency of the nanoparticles in the subject; the control characteristic parameter is the control clearance efficiency of the nanoparticles in the control group; and if the clearance efficiency is significantly different from the control clearance efficiency, then it indicates kidney dysfunction or injury.

In some embodiments of any one of the above aspects, the characteristic parameter is the emission spectrum of the nanoparticles in the urine sample of the subject; the control characteristic parameter is the control emission spectrum of the nanoparticles in the urine sample of the control group; and if the emission spectrum is significantly different from the control emission spectrum, then it indicates kidney dysfunction or injury.

In some embodiments of any one of the above aspects, the kidney dysfunction is caused by drug-induced nephrotoxicity, an autoimmune disease, kidney failure, chronic kidney disease, cystic kidney disease, kidney inflammatory disease, kidney fibrosis, autosomal dominant polycystic kidney disease, an immuno-oncological treatment, or a combination thereof.

In some embodiments of any one of the above aspects, the autoimmune disease is lupus.

In some embodiments of any one of the above aspects, the kidney inflammatory disease is lupus nephritis.

In some embodiments of any one of the above aspects, the kidney injury is induced by virus infection.

In some embodiments of any one of the above aspects, the administration is intravenous, intraperitoneal, subcutaneous, or intraarterial.

In some embodiments of any one of the above aspects, the second period of time is about 1 hour to 30 days for acute kidney disease, or about two weeks to 12 months for chronic kidney disease.

### BRIEF DESCRIPTION OF THE DRAWINGS

FIG. 1 is a series of fluorescence images of terminal deoxynucleotidyl transferase dUTP nick end labeling (TUNEL) assay analysis of kidney tissues, which were obtained from mice receiving cisplatin (15 mg/kg, intraperitoneal injection) or phosphate buffered saline (“Control”). The TUNEL assay analysis revealed two different levels of cell apoptosis in the kidney, corresponding to two different stages of cisplatin-induced acute kidney injury. The apoptotic cells, labeled by FITC, were pointed by arrows. The nuclei were stained by DAPI. Stage I was an “early stage” where apoptosis was identified in the kidney tissues of cisplatin-injected mice, in contrast to the negligible apoptosis in the normal kidneys of control group. Stage II showed significantly enhanced cell apoptosis than Stage II. Scale bar, 500 μm for all the three images.

FIG. 2 is a graph showing the levels of blood urea nitrogen (BUN) of mice in two different stages of cisplatin-induced kidney injury and control group having normal kidney function. Mice receiving phosphate buffered saline served as control. N=4 for each group, *P<0.05; ns, no significant difference, P>0.05.

FIG. 3 is a graph showing the levels of serum creatinine of mice in two different stages of cisplatin-induced kidney injury and control group having normal kidney function. Mice receiving phosphate buffered saline served as control. N=4 for each group, *P<0.05; ns, no significant difference, P>0.05.

FIG. 4 is a graph showing the amount of renal clearable glutathione-coated gold nanoparticles (GS-AuNPs) that were excreted in urine within 1 hour post intravenous injection (namely “1-h renal clearance”). Mice in two different stages of cisplatin-induced kidney injury were studied. Mice receiving phosphate buffered saline served as control. % ID, percent of injected dose; N=4 for each group, *P<0.05.

FIG. 5 is a graph showing the blood concentration of renal clearable glutathione-coated gold nanoparticles (GS-AuNPs) at 1 hour post intravenous injection. Mice in two different stages of cisplatin-induced kidney injury were studied. Mice receiving phosphate buffered saline served as control. % ID/g, percent of injected dose per gram of blood; N=4 for each group, *P<0.05.

FIG. 6 are graphs showing the amount of renal clearable glutathione-coated gold nanoparticles (GS-AuNPs) in the left and right kidneys at 1 hour post intravenous injection. Mice in two different stages of cisplatin-induced kidney injury were studied. Mice receiving phosphate buffered saline served as control. % ID/g, percent of injected dose per gram of blood; N=4 for each group, *P<0.05.

FIG. 7 is a graph showing the representative emission spectra of GS-AuNPs before being injected (“Pre-Injection”) and being excreted into urine of mice. Control group and cisplatin model were studied.

FIG. 8 is a graph showing the changes of urine protein levels of an MRL-lpr lupus mouse during growth from age of 10 to 14 weeks. Normal level values were obtained from four control MRL mice at age of 8 weeks.

FIG. 9 is a graph showing the changes of 1-hour renal clearance efficiencies of GS-AuNPs (5 mg/kg, after intravenous injection) of an MRL-lpr lupus mouse during growth from age of 10 to 14 weeks. The changes of urine protein levels of this lupus mouse are shown in FIG. 8. Normal level values were obtained from four control MRL mice at age of 8 weeks.

FIG. 10 is a graph showing the urine protein levels of three MRL-lpr lupus mice during growth from age of 10-14 weeks. Normal level values were obtained from control MRL mice at age of 8 weeks. *P<0.05, T test.

FIG. 11 is a graph showing the 1-hour renal clearance efficiencies of 5 mg/kg GS-AuNPs after intravenous injection of three MRL-lpr lupus mice during growth from age of 10-14 weeks. The changes of urine protein levels of these lupus mouse are shown in FIG. 10. Normal level values were obtained from control MRL mice at age of 8 weeks. *P<0.05, T test.

FIG. 12 is a graph showing the levels of urine protein of lupus model and control group at the age of 8 weeks. Lupus model was MRL/MpJ-Faslpr/J mouse (also known as MRL-lpr), a well-established model of systemic lupus erythematosus (SLE). Control group was MRL/MpJ mouse, also known as MRL. Left, proteinuria score of the urine samples was measured using Albustix® reagent strips for analysis of urine protein. Grades of proteinuria were scored as follows: 0=none, 1=trace, 1.5=trace˜30 mg/dl, 2=30 mg/dl, 3=100 mg/dl, 4=300 mg/dl, and 5=>2,000 mg/dl. N=11 for lupus model (urine collection for the tests was failed in one mouse); N=4 for control mice; ns, no significant difference, P>0.05.

FIG. 13 is a graph showing the difference between lupus model and control group at the age of 8 weeks in the decrease percentage of blood concentration of renal clearable GS-AuNPs at 1 h compared to that at 5 min (=[(blood concentration at 5 min−blood concentration at 1 h)/ blood concentration at 5 min]×x 100%). N=12 for lupus model; N=4 for control mice; *P<0.05.

FIG. 14 is a flow diagram of an embodiment of a method for evaluating the function of a live kidney, the method being further applicable to diagnose a dysfunction of a live kidney.

FIG. 15 is a graph showing the amount of renal clearable glutathione-coated gold nanoparticles (GS-AuNPs) that were excreted in urine of normal mice and mice with glomerular injury within 1 hour post intravenous injection (namely “1-h renal clearance”). % ID, percent of injected dose; N=4 for control group, N=2 for glomerular injury, *P<0.05.

FIG. 16 are graphs showing the urine protein levels of normal mice and mice with glomerular injury. % ID, percent of injected dose; N=4 for control group, N=2 for glomerular injury.

### DETAILED DESCRIPTION

The present disclosure relates to methods that utilize renal clearable nanoparticles to diagnose, evaluate, and/or monitor kidney function in a subject. After the renal clearable nanoparticles are administered to a subject, at least a portion of the nanoparticles in the bloodstream is removed by the kidney as part of the urine. These nanoparticles, in a blood sample or a urine sample, can produce one or more quantifiable characteristic parameters, which can be significantly different in a subject with normal kidney function versus a subject with kidney dysfunction or kidney injury. Therefore, these nanoparticles can be used as an exogenous marker to indicate the health of the kidney. Notably, the methods described herein can detect kidney dysfunction or injury even in situations where current serum or urine biomarkers fail to do so, thereby permitting early detection of kidney dysfunction or injury.

The nanoparticles can be made of one or more materials. In some embodiments, the nanoparticles can comprise gold, silver, copper, platinum, palladium, silica, carbon, silicon, iron oxide, FeS, a semiconductor quantum dot, an organic material, or a combination thereof. Examples of semiconductor quantum dots include, but are not limited to, CdTe, CdSe, CdS, and CuS.

In some embodiments, the nanoparticles can comprise an organic material that can be eliminated through the kidneys and have sizes in the range of about 0.3 nm to 10 nm or a molecular weight in the range of 500 to 20,000 Dalton. The organic nanoparticles can be labeled with dyes or other probes.

In some embodiments, the nanoparticles can comprise an organic material with an average size below about 8 nm, such as PEGylated organic dyes and zwitterionic organic dyes.

In some embodiments, the nanoparticles can comprise a core and optionally a surface coating surrounding the core. In some embodiments, the nanoparticles have an average core size of no more than about 6 nm, no more than about 5.5 nm, no more than about 5 nm, no more than about 4.5 nm, no more than about 4 nm, no more than about 3.5 nm, no more than about 3 nm, or no more than about 2.5 nm. In some embodiments, the nanoparticles have an average core size of at least about 0.5 nm, at least about 0.8 nm, or at least about 1 nm.

Combinations of the above-referenced ranges for the average core size are also possible (e.g., at least about 0.5 nm to no more than about 6 nm, or at least about 0.5 nm to no more than about 2.5 nm), inclusive of all values and ranges therebetween. In some embodiments, the nanoparticles are gold nanoparticles having an average core size of at least about 0.5 nm to no more than about 2.5 nm.

In some embodiments, the nanoparticles have an average hydrodynamic diameter of no more than about 10 nm, no more than about 9 nm, no more than about 8 nm, no more than about 7 nm, no more than about 6 nm, no more than about 5 nm, or no more than about 4 nm. In some embodiments, the nanoparticles have an average hydrodynamic diameter of at least about 1 nm, at least about 1.5 nm, or at least about 2 nm, at least about 2.5 nm.

Combinations of the above-referenced ranges for the average hydrodynamic diameter are also possible (e.g., at least about 1 nm to no more than about 10 nm, or at least about 1 nm to no more than about 4 nm), inclusive of all values and ranges therebetween.

In some embodiments, the core of each nanoparticle can comprise no more than about 41,000 atoms, no more than about 35,000 atoms, no more than about 30,000 atoms, no more than about 25,000 atoms, or no more than about 9,000 atoms. In some embodiments, the core of each nanoparticle can comprise at least about 2 atoms, at least about 25 atoms, at least about 30 atoms, at least about 35 atoms, or at least about 40 atoms.

Combinations of the above-referenced ranges for the number of atoms for the core of each nanoparticle are also possible (e.g., at least about 2 to no more than about 41,000, or at least about 25 to no more than about 35,000), inclusive of all values and ranges therebetween. In some embodiments, the nanoparticles comprise between 10 and 650 metal atoms with an average core size of between 0.5 nm and 2.5 nm.

The surface coating can comprise one or more types of ligands. In some embodiments, the ligand can be selected from the group consisting of glutathione, thiol-functionalized polyethylene glycol (PEG), cysteamine, cysteine, homocysteine, a dipeptide containing cysteine, a dipeptide containing homocysteine, a peptide having more than three amino acids, and a combination thereof. In some embodiments, the dipeptide containing cysteine includes cysteine-glycine or cysteine-glutamic acid. In some embodiments, the dipeptide containing homocysteine includes homocysteine-glycine or homocysteine-glutamic acid.

In some embodiments, the thiol-functionalized PEG has a molecular weight in the range of about 150 to 10,000 Dalton.

In some embodiments, the ligand can be conjugated with a fluorescent dye.

The nanoparticles can fluoresce on their own or due to the fluorescent dye. In some embodiments, the nanoparticles can fluoresce in the visible range, e.g., in a range of 500 to 850 nm. In some embodiments, the nanoparticles can fluoresce in the near-infrared range, e.g., in a range of 1000 to 1700 nm.

The nanoparticles can be in the form of an aqueous solution or suspension. The aqueous solution or suspension can further include an agent for preventing the nanoparticles from forming aggregates.

In one aspect, the present disclosure provides a method of evaluating kidney function of a subject, the method comprising: (a) administering to the subject a first plurality of nanoparticles having a first dose; (b) collecting a urine sample and/or a blood sample from the subject after a first period of time after the administration; (c) characterizing the nanoparticles in the urine sample and/or the blood sample with a measurement process to obtain a characteristic parameter; and (d) comparing the characteristic parameter with a control characteristic parameter measured for a control group having normal kidney function, thereby evaluating the kidney function.

In another aspect, the present disclosure provides a method of evaluating kidney function of a subject, the method comprising: (c) characterizing a first plurality of nanoparticles in a urine sample and/or a blood sample collected after a first period of time from the subject administered with the first plurality of nanoparticles, with a measurement process to obtain a characteristic parameter; and (d) comparing the characteristic parameter with a control characteristic parameter measured for a control group having normal kidney function, thereby evaluating the kidney function.

In some embodiments, the control characteristic parameter can be measured by: (e) administering to the control group a second plurality of the nanoparticles having a second dose; (f) collecting a urine sample and/or a blood sample from the control group after the first period of time after the administration; and (g) characterizing the nanoparticles in the urine sample and/or the blood sample with the measurement process to obtain the control characteristic parameter.

If the characteristic parameter is measured in a blood sample of the subject, then for comparison purposes, the control characteristic parameter is also measured in one or more blood samples of the control group. Similarly, if the characteristic parameter is measured in a urine sample of the subject, then for comparison purposes, the control characteristic parameter is also measured in one or more urine samples of the control group.

The control group can comprise one or more subjects with normal kidney function. In some embodiments, the control group can comprise about 5 to 20, about 20 to 100, about 100 to 250, about 250 to 500, about 500 to 1,000, or a combination thereof, or more than 1,000 subjects with normal kidney function.

In some embodiments, the administration is intravenous, intraperitoneal, subcutaneous, or intraarterial. In some embodiments, the nanoparticles are injected intravenously.

The dose (e.g., a first dose, a second dose) can depend on the type of nanoparticles and/or the technique used in obtaining the characteristic parameter. For example, for inductively coupled plasma mass spectroscopy, the dose can be in the range of about 10⁻⁹ mmol/kg to about 10⁻³ mmol/kg; for magnetic resonance imaging, the dose can be in the range of about 0.01 mmol/kg to about 10 mmol/kg; for radioactive detection, the dose can be in the range of about 10⁻¹ mmol/kg to about 10⁻⁶ mmol/kg.

In some embodiments, the first dose is the same as the second dose. In some embodiments, the first dose is different from the second dose.

After the nanoparticles are administered to a subject, they enter the bloodstream of the subject. After a certain period of time, at least a portion of the nanoparticles in the bloodstream is removed by the kidney as part of the urine. Kidney removal of the nanoparticles may be through glomerular filtration, renal tubular reabsorption, renal tubular secretion, or combinations thereof.

The nanoparticles can interact with the blood, kidney, and/or urine of a subject with normal kidney function in a manner different from that of a subject with kidney dysfunction or injury, thereby resulting in a significant difference in the characteristic parameter. Accordingly, in some embodiments, the method further comprises indicating kidney dysfunction or injury when the characteristic parameter is significantly different from the control characteristic parameter or reference value. In some embodiments, the kidney dysfunction or injury is early stage. In some embodiments, the kidney dysfunction or injury is late stage.

The measurement process used to obtain the characteristic parameter or control characteristic parameter includes one or more processes selected from the group consisting of: measuring a concentration of the nanoparticles, measuring an amount of the nanoparticles, measuring clearance efficiency, analyzing a composition of ligands on the nanoparticle surface, measuring size distribution of the nanoparticles, measuring an absorption spectrum of the nanoparticles, measuring an emission spectrum of the nanoparticles, measuring an excitation spectrum of the nanoparticles, measuring a photoacoustic signal of the nanoparticles, measuring radioactivity of the nanoparticles, or a combination thereof.

In some embodiments, the measurement process includes inductively coupled plasma mass spectrometry (ICP-MS). In some embodiments, the measurement process includes inductively coupled plasma optical emission spectroscopy (ICP-OES).

Related to the measurement process, the characteristic parameter or control characteristic parameter can be selected from the group consisting of: a concentration of the nanoparticles, an amount of the nanoparticles, clearance efficiency, a composition of ligands on the nanoparticle surface, one type of surface ligand, size distribution of the nanoparticles, surface charge of the nanoparticles, an absorption spectrum of the nanoparticles, an emission spectrum of the nanoparticles, an excitation spectrum of the nanoparticles, a photoacoustic signal of the nanoparticles, radioactivity of the nanoparticles, and a combination thereof.

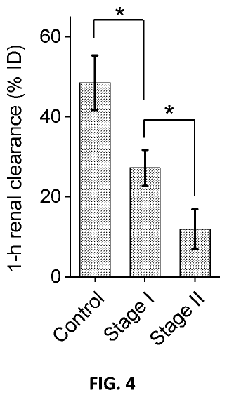

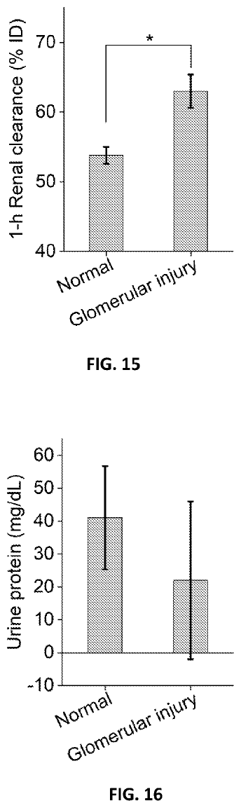

When there is kidney dysfunction or injury, the clearance efficiency of the nanoparticles in the urine is significantly different as compared to a control with normal kidney function. For example, see FIG. 4 and FIG. 15. Without wishing to be bound by theory, this is because the clearance efficiency depends on the mechanism of kidney dysfunction or injury. Accordingly, in some embodiments, the characteristic parameter is the clearance efficiency of the nanoparticles in the urine sample of the subject; the control characteristic parameter is the control clearance efficiency of the nanoparticles in the urine sample of the control group; and if the clearance efficiency is significantly different from the control clearance efficiency, then it indicates kidney dysfunction or injury.

In some embodiments, the clearance efficiency is significantly less than the control clearance efficiency, e.g., the clearance efficiency is about 95%, about 90%, about 85%, about 80%, about 75%, about 70%, about 65%, about 60%, about 55%, about 50%, about 45%, about 40%, about 35%, about 30%, about 25%, about 20%, about 15%, or about 10% of the control clearance efficiency.

In some embodiments, the clearance efficiency is significantly greater than the control clearance efficiency, e.g., the clearance efficiency is about 105%, about 110%, about 115%, about 120%, about 125%, about 130%, about 135%, about 140%, about 145%, about 150%, about 155%, about 160%, about 165%, about 170%, about 175%, about 180%, about 185%, or about 190% of the control clearance efficiency.

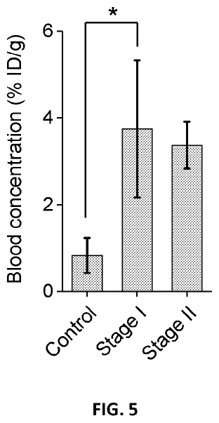

When there is kidney dysfunction or injury, the concentration of the nanoparticles in the blood is significantly different as compared to a control with normal kidney function. For example, see FIG. 5. Accordingly, in some embodiments, the characteristic parameter is the concentration of the nanoparticles in the blood sample of the subject; the control characteristic parameter is the control concentration of the nanoparticles in the blood sample of the control group; and if the concentration is significantly different from the control concentration, then it indicates kidney dysfunction or injury.

In some embodiments, the concentration of the nanoparticles is significantly less than the control concentration, e.g., the concentration of the nanoparticles is about 95%, about 90%, about 85%, about 80%, about 75%, about 70%, about 65%, about 60%, about 55%, about 50%, about 45%, about 40%, about 35%, about 30%, about 25%, about 20%, about 15%, or about 10% of the control concentration.

In some embodiments, the concentration of the nanoparticles is significantly greater than the control concentration, e.g., the concentration of the nanoparticles is about 105%, about 110%, about 115%, about 120%, about 125%, about 130%, about 135%, about 140%, about 145%, about 150%, about 155%, about 160%, about 165%, about 170%, about 175%, about 180%, about 185%, or about 190% of the control concentration.

During circulation in the bloodstream, the nanoparticle surface may acquire a different amount of biological thiols in the blood of a subject with kidney dysfunction or injury as compared to a control with normal kidney function. As a result, the emission spectrum of the nanoparticles excreted from a dysfunctional or injured kidney can be significantly different from that of the nanoparticles excreted from a kidney with normal function.

In some embodiments, instead of relying on a control characteristic parameter, a reference value can be used for comparison purposes. The reference value can be obtained from a database, which includes the characteristic parameters of controls having normal kidney function and statistical averages thereof. In some embodiments, the statistical average is an arithmetic mean, a geometric mean, or a harmonic mean. In some embodiments, the reference value can be a range.

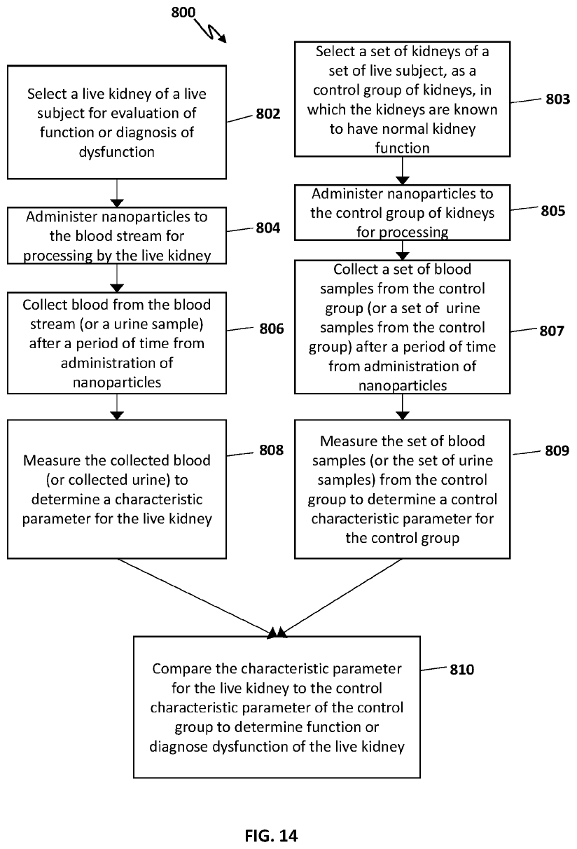

Referring to FIG. 14, an embodiment of the disclosure is shown in method 800, a method for evaluating kidney function. The method begins at step 802 by selecting a live kidney from a live subject for evaluation or diagnosis and then continues, at step 804, by administering nanoparticles to the blood stream of the live subject via a blood vessel connected to the live kidney so that the live kidney processes the blood stream. At step 806, a blood sample is collected from the blood stream (processed by the live kidney) after a first period of time. Alternatively, at step 806, a urine sample (processed by the live kidney) is collected from the live subject in a second period of time. The period of time for collecting blood or for collecting urine can be the same or different.

After collection, at step 808, the blood sample or the urine sample are analyzed using an appropriate measurement process to obtain a characteristic parameter for the live kidney under test. A control characteristic parameter is measured for a control group of kidneys known to have normal kidney function as shown in steps 803, 805, 807 and 809.

Then, at step 810, the measured characteristic parameter is compared to the control characteristic parameter to evaluate and determine the function (or diagnose the dysfunction or injury) of the live kidney.

It should be understood that for the control group, a set of kidneys, known to be functioning normally are selected (step 803). Then, at step 805, nanoparticles are administered to a blood vessel connected to at least one kidney of the control group. At step 807, at least one of: a blood sample or a urine sample is collected after the first period of time after administering the nanoparticles (the same first period of time as in step 806) or collecting total urine sample within the second period of time after administering the nanoparticles (the same second period of time as in step 806), wherein the blood sample and the urine sample are processed by the at least one kidney. At step 809, the nanoparticles in the urine sample and the blood sample are characterized using the same type of measurement process as in step 808 to obtain the control characteristic parameter for the at least one kidney.

In some embodiments, the method 800 may begin at step 808, while steps 802, 804, and 806 may be conducted separately from step 808 and or step 810, which may be carried out by a medical professional, such as a doctor or a nurse, or the subject, i.e., the methods of the present disclosure comprises the diagnostic steps 808 and/or 810, and may optionally comprise steps 802, 804, and 806. Similarly, steps 803, 805, and 807 may be conducted separately from step 809 and/or step 810, which may be carried out by a medical professional, such as a doctor or a nurse, or the subject.

Method 800 as applied to diagnose the dysfunction or injury of a live kidney is especially sensitive to detecting early stages of kidney dysfunction when renal function biomarkers such as BUN, serum creatinine and urine proteins remain in the normal range.

The dysfunction of the live kidney is not limited to acute kidney disease such as drug-induced nephrotoxicity. The methods described herein are also applicable when the dysfunction of the kidney is caused by at least one of: an autoimmune disease such as lupus; chronic kidney disease that is related to diabetes, glomerulonephritis, or high blood pressure; kidney failure; kidney inflammatory disease, such as lupus nephritis; kidney fibrosis; autosomal dominant polycystic kidney disease; an immuno-oncological treatment; cystic kidney disease; or a combination thereof.

In some embodiments, the immuno-oncological treatment is an immunotherapy.

In some embodiments, the kidney injury is induced by an infection, e.g., a viral infection, a bacterial infection, or a parasite infection.

In some embodiments, the kidney injury is caused by ureter injury and other related ureter trauma, such as ureteral obstruction and ureteral discontinuity.

Further to the measurement process, blood collection may be performed at one time point (such as 1 hour or 24 hours post administration), at two time points (such as 5 minutes and 1 hour post administration), or at multiple time points (such as 5 minutes, 1 hour, and 24 hours post administration). In some embodiments, the blood sample is collected about 5 minutes to 1 hour post administration.

Further to the measurement process, the urine sample is collected within a period of time, such as about 30 minutes to 1 hour or about 30 minutes to 24 hours post administration.

In some embodiments, the urine sample may be collected from the ureter of a donor kidney for transplant.

In the step of comparing, statistical analysis is applied to determine whether there are statistically significant differences between the two data sets. A statistically significant difference in the characteristic parameter (for the live kidney under test), when compared to the control characteristic parameter, indicates a renal dysfunction or injury.

The nanoparticles described herein can also be used to monitor over time the kidney function of a subject—particularly a subject with or susceptible to kidney dysfunction or injury.

Accordingly, in one aspect, the present disclosure provides a method of monitoring kidney function of a subject, the method comprising: (a) administering to the subject a first plurality of nanoparticles having a first dose; (b) collecting a first urine sample and/or a first blood sample from the subject after a first period of time after the administration of step (a); (c) characterizing the nanoparticles in the first urine sample and/or the first blood sample with a measurement process to obtain a first characteristic parameter; (d) after a second period of time after step (b), administering to the subject a second plurality of the nanoparticles having a second dose; (e) collecting a second urine sample and/or a second blood sample from the subject after a third period of time after the administration of step (d); (f) characterizing the nanoparticles in the second urine sample and/or the second blood sample with the measurement process to obtain a second characteristic parameter; and (g) comparing the first characteristic parameter with the second characteristic parameter, thereby monitoring the kidney function over time.

In some embodiments, the first dose is the same as the second dose. In some embodiments, the first dose is different from the second dose.

In yet another aspect, the present disclosure provides a method of monitoring kidney function of a subject, the method comprising: (c) characterizing a first plurality of nanoparticles in a first urine sample and/or a first blood sample collected after a first period of time from the subject administered with the first plurality of nanoparticles, with a measurement process to obtain a first characteristic parameter; (d) after a second period of time after step (c), characterizing a second plurality of nanoparticles in a second urine sample and/or a second blood sample collected after a third period of time from the subject administered with the second plurality of nanoparticles, with the measurement process to obtain a second characteristic parameter; and (e) comparing the first characteristic parameter with the second characteristic parameter, thereby monitoring the kidney function over time.

In some embodiments, the third period of time is the same as the first period of time. In some embodiments, the third period of time is different from the first period of time.

In some embodiments of any one of the above aspects, the first period of time is about 30 minutes to 2 hours for the urine sample, or about 5 mins to 1 hour for the blood sample.

In some embodiments of any one of the above aspects, the third period of time is about 30 minutes to 24 hours for the urine sample, or about 5 mins to 24 hours for the blood sample.

The second period of time and the frequency of monitoring depend on the severity of the kidney dysfunction or injury. For acute kidney disease, the second period of time can be about 1 hour to several weeks, e.g., about 1 hour to 30 days, about 1 hour to 4 weeks, about 1 hour to 3 weeks, about 1 hour to 14 days, about 1 hour to 7 days, about 4 hours to 30 days, about 4 hours to 14 days, or about 4 hours to 7 days. For chronic kidney disease, the second period of time can be about several weeks to several months, e.g., about 2 weeks to 12 months, about 2 weeks to 6 months, about 2 weeks to 5 months, about 2 weeks to 4 months, about 2 weeks to 3 months, or about 1 month to 3 months.

For temporal monitoring, there can be more than 2 time points of measurement, e.g., at least 3 time points, at least 4 time points, or at least 5 time points.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

While various embodiments have been described and illustrated herein, a variety of other means and/or structures for performing the function and/or obtaining the results and/or one or more of the advantages described herein, and each of such variations and/or modifications are possible. More generally, all parameters, dimensions, materials, and configurations described herein are meant to be examples and that the actual parameters, dimensions, materials, and/or configurations will depend upon the specific application or applications for which the disclosure is used. It is to be understood that the foregoing embodiments are presented by way of example only and that other embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”. “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily to including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one,

B (and optionally including other elements); etc.

As used herein, the term “about” means a range of values that are similar to the stated reference value. In certain embodiments, the term “about” refers to a range of values that fall within 10 percent or less (e.g., 10%, 9%, 8%, 7%, 6%, 5%, 4%, 3%, 2%, or 1%) of the stated reference value.

As used herein, the term “significantly” means at least 5%, at least 10%, at least 15%, at least 20%, at least 25%, at least 30%, at least 35%, at least 40%, at least 45%, or at least 50%.

As used herein, the term “renal clearable,” when applied to nanoparticles or nanomaterials, means that the nanoparticles or nanomaterials have a 1-hour or a 2-hour renal clearance efficiency of at least 5%, at least 10%, at least 15%, at least 20%, at least 25%, at least 30%, at least 35%, at least 40%, at least 45%, or at least 50% of the injected dose. In some embodiments, the renal clearable nanoparticles or nanomaterials have a 1-hour renal clearance efficiency of at least 5%, at least 10%, at least 15%, at least 20%, at least 25%, at least 30%, at least 35%, at least 40%, at least 45%, or at least 50% of the injected dose. In some embodiments, the renal clearable nanoparticles or nanomaterials have a 1-hour renal clearance efficiency in the range of about 5% to 100% of injected dose. In some embodiments, the renal clearable nanoparticles or nanomaterials have a 1-hour or a 2-hour renal clearance efficiency of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the injected dose. In some embodiments, the renal clearable nanoparticles or nanomaterials have a 1-hour renal clearance efficiency of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the injected dose.

As used herein, the term “clearance efficiency” means the amount of nanoparticles excreted into urine at a certain time point post administration divided by the amount of nanoparticles administered to the subject. As such, clearance efficiency can be time dependent.

As used herein, the term “kidney function” means generally the functional status of the kidney. The term “kidney function” can be used to describe the function of a healthy kidney (healthy kidney function) or the function of a kidney that is impaired or injured or a kidney having a disease or disorder (impaired kidney function). In one embodiment, kidney function is represented by the excretory capacity of the kidney.

As used herein, the term “kidney dysfunction” means that the function of the kidney is below the function of a healthy kidney, including about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the function of a healthy kidney, e.g., the kidney when the subject is healthy, or of the average kidney function of a group of healthy subjects.

As used herein, the term “early stage,” when applied to kidney dysfunction, injury, or disease, means a stage when the kidney dysfunction, injury, or disease cannot be identified by current kidney injury/function biomarkers, such as serum biomarkers (e.g., blood urea nitrogen or serum creatinine) and urine biomarkers (e.g., urine protein, urine protein-to-urine creatinine ratio, or urine albumin-to-urine creatinine ratio).

As used herein, the term “late stage,” when applied to kidney dysfunction, injury, or disease, means a stage when the kidney dysfunction, injury, or disease can be identified by current kidney injury/function biomarkers, such as serum biomarkers (e.g., blood urea nitrogen or serum creatinine) and urine biomarkers (e.g., urine protein, urine protein-to-urine creatinine ratio, or urine albumin-to-urine creatinine ratio).

As used herein, the term “dose” means the amount of nanoparticles that are administered to a subject. A dose can have a variety of units, such as mg/kg (milligram of nanoparticles per kilogram of body weight) and mmol/kg (millimole of nanoparticles per kilogram of body weight).

As used herein, the term “subject” means a human or other vertebrate animal. In some embodiments, the subject is a human having kidney dysfunction, injury, or disease. In some embodiments, the subject is a human susceptible to having kidney dysfunction, injury, or disease. In some embodiments, the subject is a human suffering from a disease or disorder that is prone to cause kidney dysfunction, injury, or disease, such as an autoimmune disease or disorder (e.g., rheumatoid arthritis, lupus, Inflammatory bowel disease, multiple sclerosis, Type 1 diabetes mellitus, Guillain-Barre syndrome, chronic inflammatory demyelinating polyneuropathy, psoriasis, Graves' disease, Hashimoto's thyroiditis, myasthenia gravis, or vasculitis). In some embodiments, the subject is a human receiving a treatment for a disease or disorder, including chemotherapy or an immune-oncological treatment. Examples of chemotherapy drugs include, but are not limited to, alkylating agents, nitrosoureas, anti-metabolites, plant alkaloids, anti-tumor antibiotics, hormonal agents, and biological response modifiers. In some embodiments, the subject is a human receiving an immuno-oncological treatment.

In the claims, as well as in the specification above, all transitional phrases such as “comprising,” “including,” “carrying,” “having,” “containing,” “involving,” “holding,” “composed of” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

The embodiments of methods and characterization data are shown by way of example in this section.

### Example 1

Gold nanomaterials have been extensively explored in disease diagnosis and treatment in vivo. They can serve as contrast imaging agents for X-ray imaging or deliver other imaging agents to the diseased organs and tissues. Due to large size or serum protein adsorption, conventional gold nanomaterials are often accumulated in the reticuloendothelial system (RES) organs (e.g., liver and spleen) for a long time after injection. For example, liver uptake of gold nanomaterials was about 15-90% ID at 24 h post injection. Gold nanoparticles are often considered biocompatible because they are relatively inert in biological environment. Although the FDA has not approved any gold-based nanomedicines, many types of gold nanoparticles are currently under preclinical development and two types of gold nanoparticles, CYT-6091 and AuroShell, have been tested in clinical trials for cancer treatment.

To further reduce the potential toxicity of gold nanoparticles related to their long-term body accumulation, a series of renal clearable gold nanoparticles were developed and their applications in disease diagnosis was conducted in the past decade. For example, renal clearable glutathione-coated gold nanoparticles (GS-AuNPs) were synthesized that show a maximum emission at 810 nm and have an average core size of 2.5 nm and hydrodynamic diameter of 3.3 nm. Each 2.5 nm GS-AuNP contains 640 gold atoms. Different from conventional gold nanomaterials, these GS-AuNPs were dominantly eliminated from the body by the kidneys. At 24 h post-intravenous injection, about 50% ID were excreted in the urine of mice or non-human primates. The GS-AuNPs were also highly biocompatible: the no-observed-adverse-effect-level (NOAEL) of these nanoparticles was measured to be >1000 mg/kg in CD-1 mice and >250 mg/kg in cynomolgus monkeys.

To test whether GS-AuNPs can serve as an exogenous marker for kidney function evaluation, the cisplatin-induced acute kidney injury model, which is one of the most widely used models for understanding of drug-induced acute kidney injury, was used. Briefly, 16 male CD-1 mice (30-35 g, 8-10 week-old) were randomly divided into two groups: (1) twelve mice received a single intraperitoneal injection of cisplatin at 15 mg/kg on Day 0, (2) four mice received a single intraperitoneal injection of phosphate-buffered saline (PBS, control group) on Day 0. Four mice receiving cisplatin were randomly selected to measure the renal clearance and plasma clearance of GS-AuNPs on Day 1, 2, 3, respectively. The control study using 4 mice receiving PBS was performed on Day 3. 183 mg/kg GS-AuNPs was intravenously injected into the mice. At 1-h post intravenous injection of GS-AuNPs, urine, blood, and kidneys were collected to measure the 1-h renal clearance, blood concentration and kidney accumulation of AuNPs with inductively coupled plasma mass spectrometry (ICP-MS). In addition, serum samples were also collected for the measurement of BUN and serum creatinine. Kidneys were processed for pathological analysis to identify structural changes of tissues. Since tubular cell apoptosis plays an essential pathogenic role for cisplatin-induced acute kidney injury, cell apoptosis was also examined by using terminal deoxynucleotidyl transferase-mediated digoxigenin-deoxyuridine nick-end labeling (TUNEL) staining.

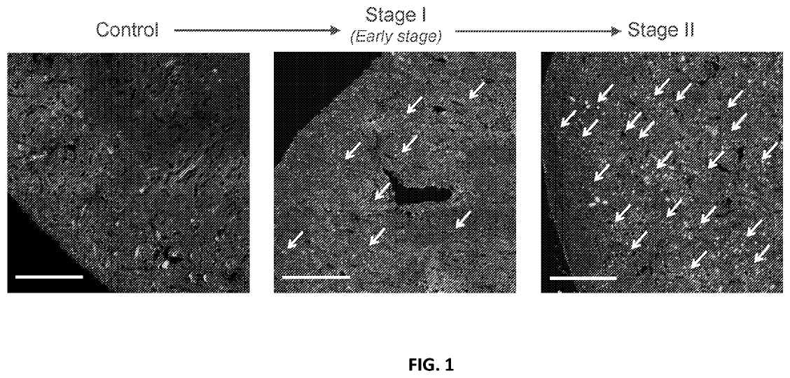

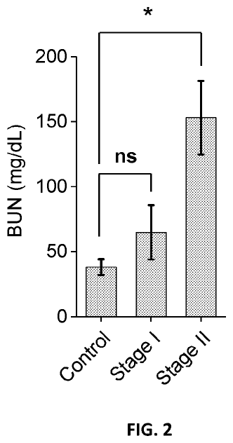

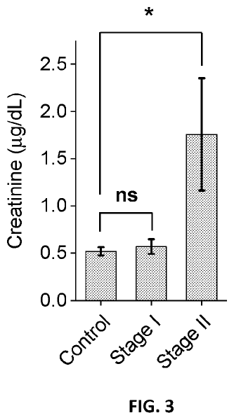

Using TUNEL assay, two different stages of the cisplatin-induced acute kidney injury were identified. Stage I was an early stage where apoptosis was identified in the kidney tissues of cisplatin-injected mice, in contrast to the negligible apoptosis in the normal kidneys of control group (FIG. 1); however, BUN and serum creatinine levels of these mice remained in the normal range obtained from control group (FIG. 2 and FIG. 3). Stage II was a mild stage that showed more pronounced apoptosis in the kidney when compared to Stage I (FIG. 1) and also exhibited a significant increase in the levels of BUN and serum creatinine than normal control (FIG. 2 and FIG. 3).

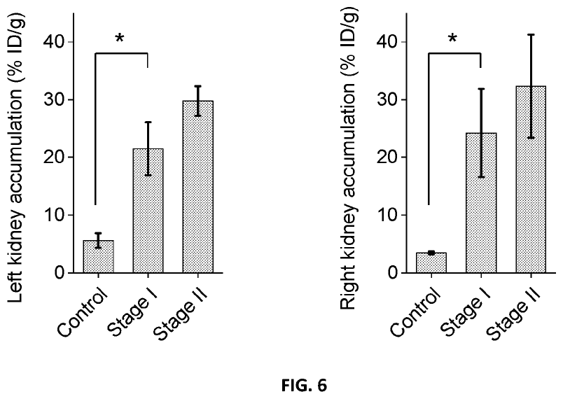

In contrast to the silence of BUN and serum creatinine at Stage I, an early stage, clearance of renal clearable GS-AuNPs was highly sensitive to the functional status of the kidney in cisplatin-induced acute kidney injury model. Compared with control group, the urine excretion of GS-AuNPs decreased and blood retention was prolonged in the cisplatin model. The 1-h urine excretion of GS-AuNPs was well correlated with the degrees of apoptosis of kidney that were assessed by TUNEL assay. Using ICP-MS, we measured the amount of gold in the urine. The 1-h renal clearance efficiency decreased from 48.5±6.8% ID in control mice with normal kidney function to 27.2±4.5% ID in mice with kidney dysfunction at an early stage when BUN and serum creatinine were silent (Stage I), and 11.9±4.9% ID in mice with mild kidney dysfunction (Stage II) (FIG. 4). Consistent with the urine test results, blood test showed that the blood concentration of GS-AuNPs was well correlated with the stages of cisplatin-induced injury. At 1 h post intravenous injection, the amount of gold in blood increased from 0.8±0.4% ID/g in control mice with normal kidney function to 3.7±1.6% ID/g in mice with kidney dysfunction at an early stage when BUN and serum creatinine were silent (Stage I), and 3.4±0.5% ID/g in mice with mild kidney dysfunction (Stage II) (FIG. 5). Moreover, a gradual increase in the kidney accumulation of GS-AuNPs was found at 1 h post injection as the kidney injury became more severely (FIG. 6). At 1 h post intravenous injection, the amounts of gold in the kidney were 5.6±1.3, 21.5±4.6, and 29.8±2.6% ID per gram of tissue (% ID/g) for control group, cisplatin model at Stage I, and cisplatin model at Stage II, respectively.

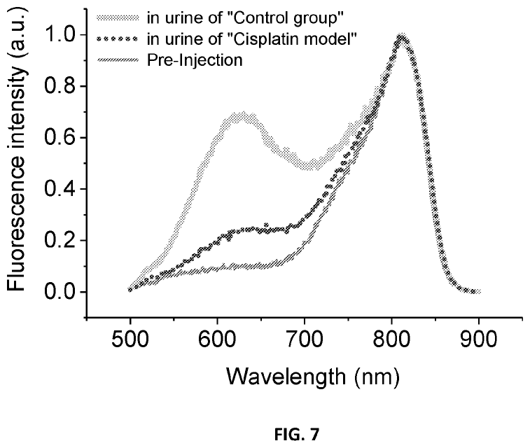

Moreover, a difference was also observed between the control group and cisplatin model in the emission spectrum of GS-AuNPs excreted into urine. The renal clearable near-infrared-emitting GS-AuNPs showed a maximum emission at 810 nm and had very weak emission at 627 nm before they were introduced into the body (“Pre-injection”, FIG. 7). After being intravenously injected to the control mice, circulating in the body, and being excreted into urine, GS-AuNPs exhibited a 6.8-fold increase of the emission intensity at 627 nm in comparison with “Pre-injection”. This dramatic increase of 627 nm suggested an enhanced surface coverage of the thiol ligands on the AuNPs, according to our previous understanding of the size-independent emission of these GS-AuNPs. The reason could be due to binding of biological thiols to the gold surface during the circulation in the body. Regarding the cisplatin model, GS-AuNPs excreted into urine only showed a 2.4-fold increase of the 627 nm emission compared with “Pre-injection”, suggesting that the level of biological thiols was significantly reduced in the cisplatin model.

### Example 2

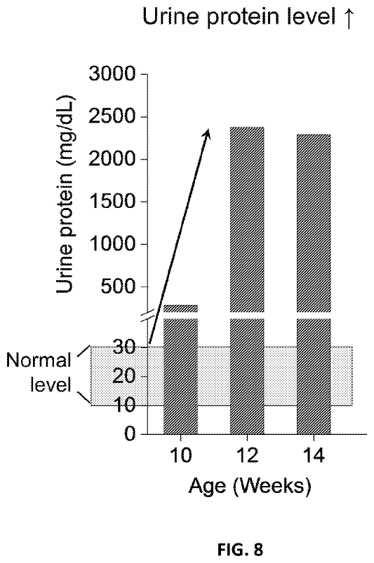

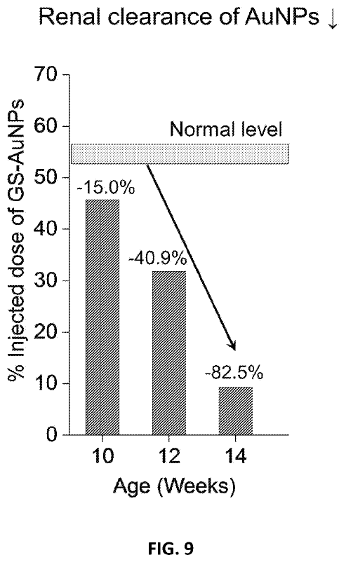

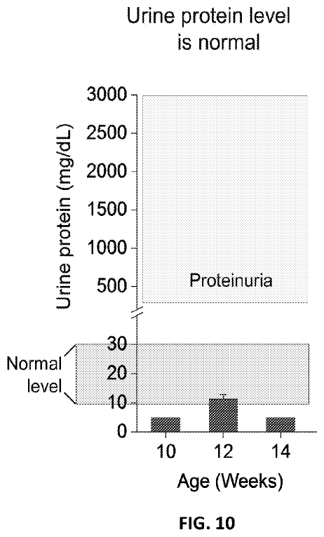

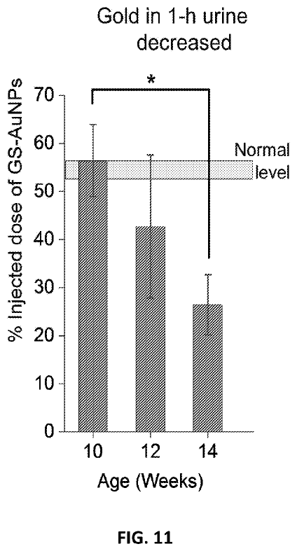

To test whether the renal clearance and plasma clearance of GS-AuNPs can be used for early diagnosis and monitoring of kidney dysfunction caused by lupus, a robust genetic mouse model of lupus—MRL/MpJ-Fas^{lpr}/J (MRL-lpr) was chosen, and MRL/MpJ mice was used as a control. The mice were purchased from The Jackson Laboratory. The MRL-lpr lupus mouse model recapitulates many clinical features of human lupus, including kidney damage, and has been used extensively in preclinical lupus research. In this genetic model of lupus, as the mice age the kidney damage increases. The urine protein level of an MRL-lpr mouse was increased by 10-times at age of 10 weeks (283 mg/dL) compared to a normal value of urine protein (10-30 mg/dL), and was further increased by 100-times at ages of 12-14 weeks (2200-2400 mg/dL), indicating the progression of lupus nephritis (FIG. 8). Consistent with the increase of urine protein level, this MRL-lpr mouse had a gradual decrease in 1-hour renal clearance of GS-AuNPs (5 mg/kg) after intravenous injection from a normal value of 53.8±1.2 percent of injected dose (% ID, n=4) to 45.7, 31.8, and 9.4% ID at age of 10, 12, and 14 weeks, corresponding to decreases of 15.0, 40.9, and 82.5% in 1-hour renal clearance compared to normal value (FIG. 9). These results indicated the renal clearance of GS-AuNPs decreased with the progression of lupus nephritis, implying that the gold amount in the urine can be used to detect the kidney damages caused by lupus. Interestingly, in three MRL-lpr lupus mice having normal urine protein levels at age of 10, 12, and 14 weeks (FIG. 10), a dramatic decrease in the 1-hour renal clearance of GS-AuNPs was observed from 56.4±7.5 to 42.7±14.9 and 26.4±6.3% ID with time during this period of time (FIG. 11).

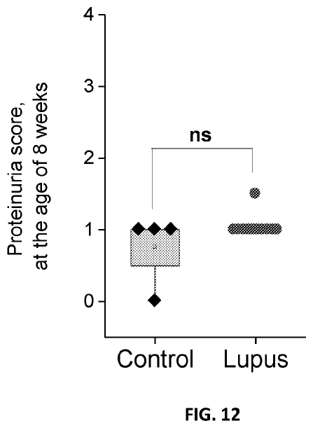

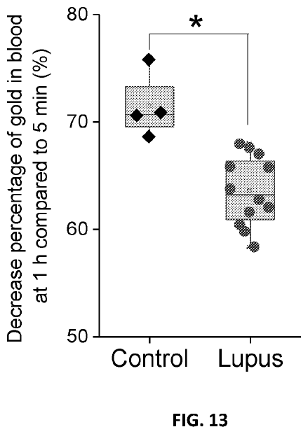

When the mice were at the age of 8 weeks, the feasibility of distinguishing the lupus model from control group using blood clearance of GS-AuNPs was also tested. On Day 0, urine samples were collected for measurement of urine protein (proteinuria). Serum samples were also collected for creatinine measurement. On Day 1, after intravenous injection of 5 mg/kg GS-AuNPs into the mice, the blood for each mouse was collected at 5 min and 1 h, and then used ICP-MS to measure gold in the urine. FIG. 12 shows that all mice (including MRL-lpr lupus model and control MRL/MpJ mice) were silent in proteinuria. However, the blood retention of GS-AuNPs was prolonged in lupus model compared to that in control MRL/MpJ group (FIG. 13). Between the MRL-lpr lupus model and control MRL/MpJ mice, a significant difference was found in the decrease percentage of blood concentration of renal clearable GS-AuNPs at 1 h compared to that at 5 min (=[(blood concentration at 5 min−blood concentration at 1 h)/blood concentration at 5 min]×100%).

### Example 3

If the glomerulus is injured, higher 1-hour renal clearance of GS-AuNPs (60-65% ID than a normal value of 53.8+1.2% ID was observed (FIG. 15) even though the urine protein levels of the diseased mice and control ones were all below 60 mg/dL and remaining in the normal range (FIG. 16).

1. A method of evaluating kidney function of a subject, the method comprising:
(a) administering to the subject a first plurality of nanoparticles having a first dose;
(b) collecting a urine sample and/or a blood sample from the subject after a first period of time after the administration;
(c) characterizing the nanoparticles in the urine sample and/or the blood sample with a measurement process to obtain a characteristic parameter; and
(d) comparing the characteristic parameter with a control characteristic parameter measured for a control group having normal kidney function, thereby evaluating the kidney function.

2. (canceled)

3. The method of claim 1 or 2, wherein the control characteristic parameter is measured by:
(e) administering to the control group a second plurality of the nanoparticles having a second dose;
(f) collecting a urine sample and/or a blood sample from the control group after the first period of time after the administration; and
(g) characterizing the nanoparticles in the urine sample and/or the blood sample with the measurement process to obtain the control characteristic parameter.

4. A method of evaluating kidney function of a subject, the method comprising:
(a) administering to the subject a first plurality of nanoparticles having a first dose;
(b) collecting a urine sample and/or a blood sample from the subject after a first period of time after the administration;
(c) characterizing the nanoparticles in the urine sample and/or the blood sample with a measurement process to obtain a characteristic parameter; and
(d) comparing the characteristic parameter with a reference value, thereby evaluating the kidney function.

5. (canceled)

6. The method of claim 1, further comprising indicating kidney dysfunction or injury when the characteristic parameter is significantly different from the control characteristic parameter.

7. A method of monitoring kidney function of a subject, the method comprising:
(a) administering to the subject a first plurality of nanoparticles having a first dose;
(b) collecting a first urine sample and/or a first blood sample from the subject after a first period of time after the administration of step (a);
(c) characterizing the nanoparticles in the first urine sample and/or the first blood sample with a measurement process to obtain a first characteristic parameter;
(d) after a second period of time after step (b), administering to the subject a second plurality of the nanoparticles having the first dose;
(e) collecting a second urine sample and/or a second blood sample from the subject after a third period of time after the administration of step (d);
(f) characterizing the nanoparticles in the second urine sample and/or the second blood sample with the measurement process to obtain a second characteristic parameter; and
(g) comparing the first characteristic parameter with the second characteristic parameter, thereby monitoring the kidney function over time.

8. (canceled)

9. The method of claim 1, wherein the nanoparticles are renal clearable.

10. The method of claim 9, wherein the nanoparticles have a 1-hour or 2-hour renal clearance efficiency in the range of 5 to 100 percent of injected dose (% ID).

11. The method of claim 9, wherein the nanoparticles comprise gold, silver, copper, platinum, palladium, silica, carbon, silicon, iron oxide, FeS, CdSe, CdS, CuS, an organic material, or a combination thereof.

12. The method of claim 1, wherein the nanoparticles are coated with a ligand selected from the group consisting of glutathione, thiol-functionalized polyethylene glycol, cysteamine, cysteine, homocysteine, a dipeptide containing cysteine, a dipeptide containing homocysteine, a peptide having more than three amino acids, and a combination thereof.

13. (canceled)

14. (canceled)

15. The method of claim 12, wherein the ligand is conjugated with a fluorescent dye.

16. The method of claim 12, wherein the ligand is glutathione.

17. The method of claim 1, wherein the nanoparticles fluoresce in a range of 500 to 850 nm.

18. The method of claim 1, wherein the nanoparticles fluoresce in a range of 1000 to 1700 nm.

19.-22. (canceled)

23. The method of claim 1, wherein the measurement process includes one or more processes selected from the group consisting of:
measuring a concentration of the nanoparticles, measuring an amount of the nanoparticles, measuring clearance efficiency, analyzing a composition of ligands on the nanoparticle surface, measuring size distribution of the nanoparticles, measuring an absorption spectrum of the nanoparticles, measuring an emission spectrum of the nanoparticles, measuring an excitation spectrum of the nanoparticles, measuring a photoacoustic signal of the nanoparticles, measuring radioactivity of the nanoparticles, measuring X-ray absorption of the nanoparticles, or a combination thereof.

24. The method of claim 23, wherein the measurement process includes inductively coupled plasma mass spectrometry (ICP-MS) or inductively coupled plasma optical emission spectroscopy (ICP-OES).

25. The method of claim 1, wherein the characteristic parameter is selected from the group consisting of: a concentration of the nanoparticles, an amount of the nanoparticles, clearance efficiency, a composition of ligands on the nanoparticle surface, one type of surface ligand, size distribution of the nanoparticles, surface charge of the nanoparticles, an absorption spectrum of the nanoparticles, an emission spectrum of the nanoparticles, an excitation spectrum of the nanoparticles, a photoacoustic signal of the nanoparticles, radioactivity of the nanoparticles, and a combination thereof.

26. The method of claim 25, wherein:
the characteristic parameter is the concentration of the nanoparticles in the blood sample of the subject;
the control characteristic parameter is the control concentration of the nanoparticles in the blood sample of the control group; and
if the concentration is significantly different from the control concentration, then it indicates kidney dysfunction or injury.

27. The method of claim 25, wherein:
the characteristic parameter is the clearance efficiency of the nanoparticles in the subject;
the control characteristic parameter is the control clearance efficiency of the nanoparticles in the control group; and
if the clearance efficiency is significantly different from the control clearance efficiency, then it indicates kidney dysfunction or injury.

28. The method of claim 25, wherein:
the characteristic parameter is the emission spectrum of the nanoparticles in the urine sample of the subject;
the control characteristic parameter is the control emission spectrum of the nanoparticles in the urine sample of the control group; and
if the emission spectrum is significantly different from the control emission spectrum, then it indicates kidney dysfunction or injury.

29. The method of claim 6, wherein the kidney dysfunction is caused by drug-induced nephrotoxicity, an autoimmune disease, kidney failure, chronic kidney disease, cystic kidney disease, kidney inflammatory disease, kidney fibrosis, autosomal dominant polycystic kidney disease, an immuno-oncological treatment, or a combination thereof.

30.-35. (canceled)

36. A method of evaluating kidney function of a subject, the method comprising:
(a) administering to the subject a first plurality of nanoparticles having a first dose; and
(b) directly measuring nanoparticle accumulation in the kidneys and comparing the accumulation in the kidneys with a control group to evaluate the kidney function.
